(19) United States
(12) Patent Application Publication
(10) Pub. No.: US 2025/0121151 A1
(43) Pub. Date: Apr. 17, 2025

(54) HIGH FLOW OXYGEN THERAPY BREATHING SYSTEMS

(71) Applicant: S.C.O.P.E. MEDICAL, INC., BRUNSWICK, OH (US)

(72) Inventors: Tosan Ugbeye, Brunswick, OH (US); Shawn Iravanchy, Rancho Palos Verdes, CA (US)

(73) Assignee: S.C.O.P.E. MEDICAL, INC., BRUNSWICK, OH (US)

(21) Appl. No.: 18/910,117

(22) Filed: Oct. 9, 2024

Related U.S. Application Data
(60) Provisional application No. 63/589,643, filed on Oct. 12, 2023.
(60) Provisional application No. 63/594,406, filed on Oct. 30, 2023.
(60) Provisional application No. 63/634,138, filed on Apr. 15, 2024.

(51) Int. Cl. A61M 16/06; A61M 16/00; A61M 16/10; A61M 16/20

(52) U.S. Cl. CPC A61M 16/0672; A61M 16/0012; A61M 16/10; A61M 16/201; A61M 16/208; A61M 2202/0007; A61M 2202/0208; A61M 2205/3334; A61M 2205/583; A61M 2230/40

(57) ABSTRACT

Provided is the OFF-VENT breathing system. The system uses novel combinations of the venturi effect, PEEP adapters, HMEs, and the adaptation of a novel oxygen delivery system for the creation of high flow therapy to maintain open airways while maintaining access to the patient's airways for medical instruments, other accessories, or to assist breathing in general. The system is designed to create high flow oxygen therapy in a manner that does not require the use of ventilators, active heating humidifiers and other expensive equipment. Embodiments described include a novel integration of the venturi system into a nasal cannula apparatus, and the novel combination of the venturi effect and a PEEP adapter into breathing systems that might include hyperinflation and Mapleson circuits.

### CROSS-REFERENCE TO RELATED APPLICATIONS

This non-provisional application claims priority to U.S. Provisional Patent Application Ser. No. 63/589,643, filed on Oct. 12, 2023, U.S. Provisional Patent Application Ser. No. 63/594,406, filed on Oct. 30, 2023, and U.S. Provisional Patent Application Ser. No. 63/634,138, filed on Apr. 15, 2024.

### I. BACKGROUND

### A. Technical Field

The present invention pertains to the field of breathing systems including masks, nasal cannulas, hyperinflation systems, Mapleson breathing circuits etc.

### B. Description of Related Art

It is desirable in medical treatment to provide high flow oxygen therapy (HFOT) that maintains open airways across a number of conditions, including respiratory failure, hypoxemia, pneumonia, management of patients with sleep apnea and obesity, sedation during medical procedures etc.

HFOT has shown a lot of promise lately in the field of respiratory care, especially after proving to be more effective at keeping patients alive at the height of the COVID-19 pandemic when patients at various stages of respiratory failure performed better on HFOT than being intubated and placed on a ventilator.

Existing HFOT systems typically require ventilators, active heating equipment, air oxygen blenders, and high-quality breathing circuits to deliver high flowing heated and humidified air to the patients' nostrils in a comfortable manner.

The performance of these systems, as well as non-invasive ventilation (NIV) systems, during the pandemic and in other areas of care, have led to more interest in incorporating them into the therapy of patients in other areas, including the perioperative management of patients with sleep apnea, and in airway support in certain anesthesia cases like deep sedation, and out of operating room (OR) cases. However, the cost of these devices, and the need for expensive equipment and personnel, currently limits their use to certain areas of care like the patient floors and the intensive care unit (ICU). For instance, out of OR anesthesia cases can occur in places without specialized equipment like ventilators and other essential personnel.

### II. SUMMARY

The present invention relates to breathing systems that utilize venturi systems and positive end-expiratory pressure (PEEP) adapters to create high flow oxygen therapy (HFOT) that maintains open airways across the aforementioned conditions, including respiratory failure, hypoxemia, pneumonia, management of patients with sleep apnea and obesity, sedation during medical procedures etc.

The breathing systems of the present invention incorporate the venturi effect, PEEP adapters, heat and moisture exchangers (HMEs), and the integration of an innovative oxygen delivery system (e.g., WOSHER, wettable oxygen shower head for enhancing respirations), to produce a system referred to herein as the OFF-VENT (offloading venturi entrainment non-invasive therapy), a system that can be used with masks, nasal cannulas, hyperinflation systems, Mapleson breathing circuits etc. The present OFF-VENT system functions by using the venturi entrainment of air to deliver a high flow during inspiration, PEEP (or, alternatively, one-way valves) to keep the airways open during expiration, and the adaptation of an innovative oxygen delivery to augment the air being entrained by the venturi system with oxygen that can be heated and humidified.

The systems of the present invention include combinations of venturi adapters and PEEP valves also including an access port for the introduction of medical instruments like Esophagogastroduodenoscopy (EGD) probes. The systems of the present invention also includes integrations of venturi adapters and PEEP valves into Mapleson breathing circuits or hyperinflation systems. Such a system can incorporate one-way valves, to create a high inspiratory flow, while using PEEP/APL valves for keeping airways stented open during expiration, and the possible inclusion of breathing bags for further airway support when needed.

The present invention also uses the entrainment of room air via the venturi effect to create HFOT, by augmenting that room air with oxygen, which can be heated, humified, and diffusely delivered in mist form through the WOSHER, and by incorporating HMEs, the OFF-VENT system is able to create the functionality of HFOT without requiring expensive equipment like ventilators and active heating humidifiers.

Provided in this disclosure is a nasal apparatus system, including a cannula having one or more oxygen inlet nozzles connected to oxygen tubing to deliver inlet oxygen towards nostrils of a patient. One ore more air entrainment windows are positioned on the cannula to support the oxygen inlet nozzle while open to the ambient environment and thereby define a flow passage for entrainment of inlet oxygen from the nozzle for mixing and diffusing with room air from the ambient environment to produce a mixed flow. One or more tapered tubes are provided downstream of the entrainment window for receiving the mixed flow from the entrainment window and having an air flow passage that is narrowed from a larger first diameter to a smaller second diameter to utilize a venturi effect to create a high velocity/pressure drop that causes the entrainment of the mixed flow to thereby deliver a high flow of oxygen to the patient's nostrils during inhalation.

In one aspect, the cannula includes first and second oxygen inlet nozzles formed with respective first and second air entrainment windows that cooperate with respective first and second tapered tubes to deliver first and second mixed flows to the nostrils of the patient. The respective first and second nozzles, windows and tubes are configured to deliver the first and second mixed flows to respective left and right nostrils of the patient.

In another aspect, the air entrainment window includes at least one support structure to support the oxygen inlet nozzle while opening to the ambient environment. In one embodiment, the tapered tube can be a discrete component with the air passage formed internally. In another embodiment, the tapered tube can be an internal component formed within a body of the cannula to define the air passage.

In still another aspect, the body of the cannula can include one or more inhalation passageways connected to one or more respective nasal pillows for contacting the nostrils during inhalation, and also including one or more exhalation passageways to allow for passage of air during exhalation. A one-way valve is provided over the exhalation passageway to allow for the expulsion of air during exhalation. The one way valve is formed of a thin layer membrane of soft material and having a fixture in place holding the one-way valve against the exhalation passageway.

In yet another aspect, the system includes a positive end-expiratory pressure (PEEP) valve configured over the exhalation passageway to allow for variable passage of less or more air during exhalation. The adjustable PEEP valve includes a spring-like mechanism with an adjustable dial to control an amount of air flowing out through the exhalation passageway. The dial is a pointer dial that cooperates with a sliding mechanism having markings to connotate the amount of air opening, for regulating exhalation airflow and positive pulmonary system pressure.

In a further aspect, the inhalation and exhalation passageways can be configured to be separate parallel passageways. The inhalation and exhalation passageways are then formed within a generally cylindrical structure divided by a bifurcation so that airflow between the inhalation and exhalation passageways are contiguous. Alternatively, the inhalation and exhalation passageways are configured to be concentric passageways. The inhalation passageway is then formed within a central cylindrical structure surrounded by a plurality of coaxial orifices to divert exhalation air.

In another further aspect, the present system can include a capnography port for connecting the nasal apparatus to a capnography machine for the monitoring of end tidal CO2 in the exhaled breath of the patient.

Other benefits and advantages of this invention will become apparent to those skilled in the art to which it pertains upon reading and understanding of the following detailed specification.

### III. BRIEF DESCRIPTION OF THE DRAWINGS

The disclosed breathing systems may take physical form in certain parts and arrangement of parts, embodiments of which will be described in detail in this specification and illustrated in the accompanying drawings which form a part hereof and wherein:

FIG. 1 is an exploded view of an exemplary embodiment of a breathing system in accordance with the present invention.

FIG. 2 is a perspective view of an exemplary embodiment of a breathing system in accordance with the present invention.

FIG. 3 is a perspective view of an exemplary embodiment of a cannular used with a breathing system in accordance with the present invention.

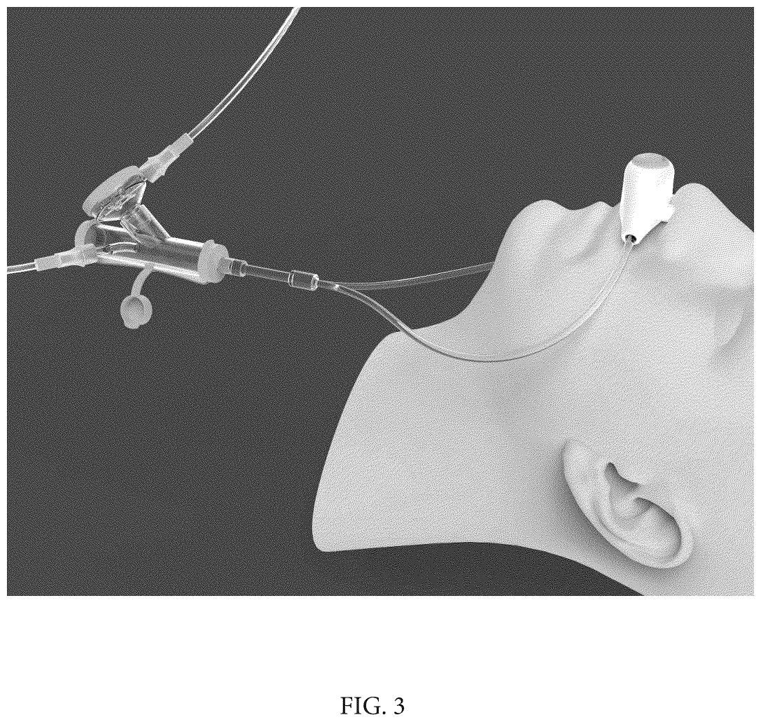

FIG. 4 is a perspective view of an exemplary embodiment of a breathing mask used with a breathing system in accordance with the present invention.

FIG. 5 is a frontal view depicting circulation through an exemplary embodiment of a breathing system in accordance with the present invention.

FIG. 6 is a cross-sectional view depicting circulation through an exemplary embodiment of a breathing system in accordance with the present invention.

FIG. 7 is an exploded view of an exemplary embodiment of a breathing system in accordance with the present invention.

FIG. 8 is a frontal view depicting circulation through an exemplary embodiment of a breathing system in accordance with the present invention.

FIG. 9 is a cross-sectional view depicting circulation through an exemplary embodiment of a breathing system in accordance with the present invention.

FIG. 10 is a reverse perspective view of an exemplary embodiment of a breathing system in accordance with the present invention.

FIG. 11 is a front perspective view of an exemplary embodiment of a breathing system in accordance with the present invention.

FIG. 11 is a front cutaway perspective view of an exemplary embodiment of a breathing system in accordance with the present invention.

FIG. 13 is a reverse perspective view of an exemplary embodiment of a breathing system in accordance with the present invention.

FIG. 14 is a front operative view of an exemplary embodiment of a breathing system being worn by a wearer in accordance with the present invention.

FIG. 15 is a side operative view of an exemplary embodiment of a breathing system being worn by a wearer in accordance with the present invention.

FIG. 16 is a perspective view of an exemplary embodiment of a breathing system in accordance with the present invention.

### IV. DETAILED DESCRIPTION

Reference is now made to the drawings wherein the showings are for purposes of illustrating embodiments of the article only and not for purposes of limiting the same, and wherein like reference numerals are understood to refer to like components.

The following detailed description is of the best currently contemplated modes of carrying out exemplary embodiments of the invention. The description is not to be taken in a limiting sense but is made merely for the purpose of illustrating the general principles of the present invention.

Embodiment of the present disclosure disclosed herein provide an OFF VENT Nasal Cannula (NC), a novel type of high flow nasal cannula (HFNC) that uses the combination of venturi entrainment of air and PEEP to perform HFOT, or a breathing system that integrates a venturi adapter and a PEEP valve, to perform the same function.

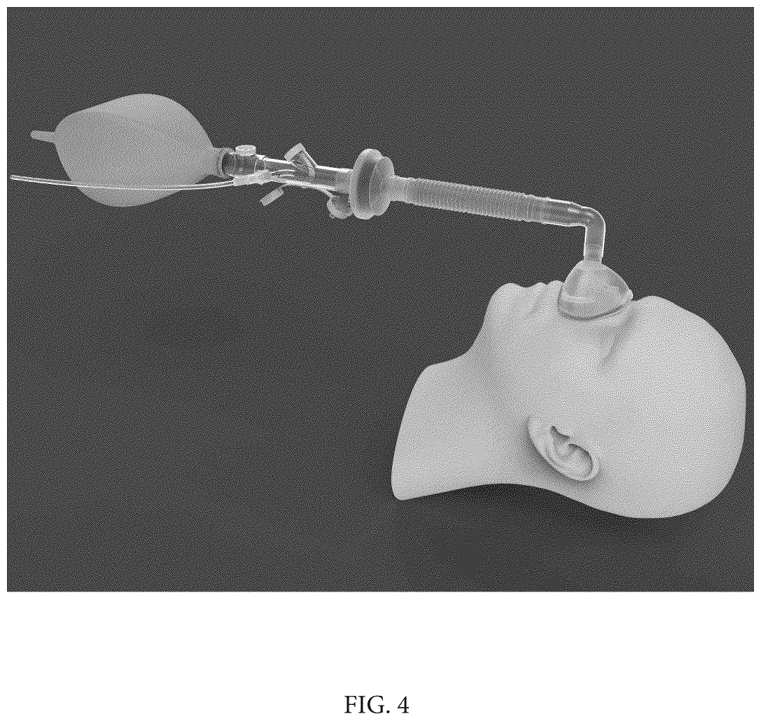

The present OFF VENT system includes a HFNC design in which a positive pressure cap connects to the filtering facepiece respirator of the present inventor's U.S. Pat. No. 11,399,912 (the disclosure of which is hereby incorporated by reference) with a self-sealing access point for the introduction of medical instruments etc. and two connectors with one housing a PEEP valve and the other housing the OFF VENT connector (a venturi adapter with an adjustable air entrainment window that can be fitted with the WOSHER connector for augmenting the entrained air with oxygen, which can be heated and humidified). A Mapleson circuit/hyperinflation breathing system can include the OFF VENT connector and a PEEP valve integrated into the breathing system and connecting to the pressure proof cap via one or multiple connectors. This breathing system might include the addition of a breathing bag for further airway support, as depicted in FIG. 4.

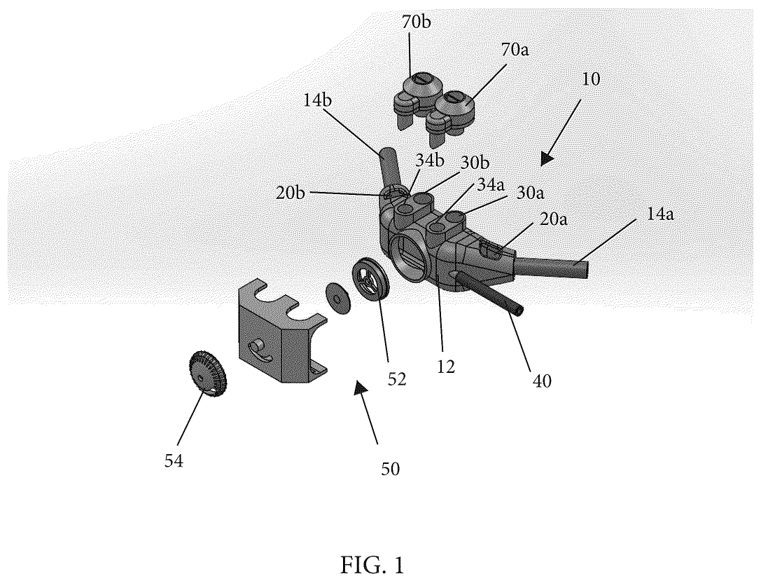

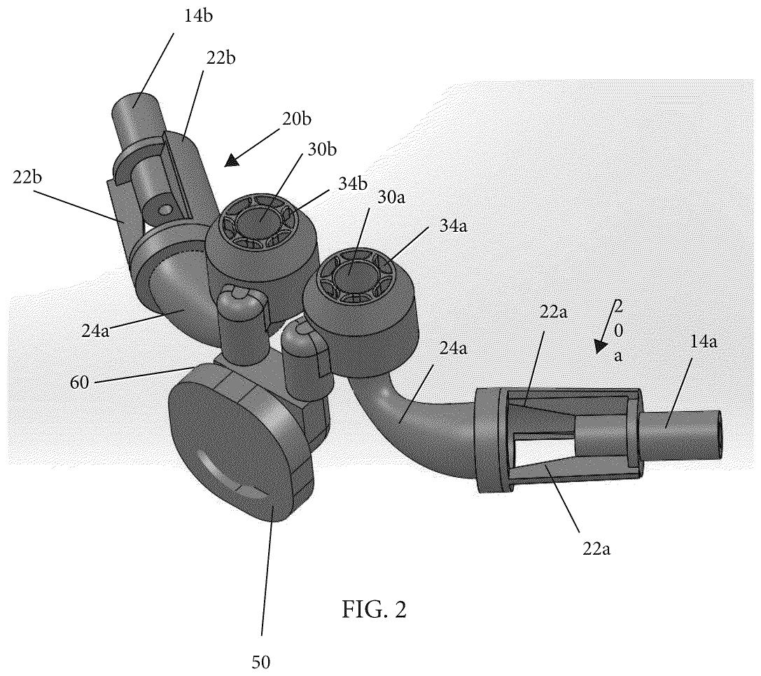

As shown in the exemplary embodiments of FIGS. 1, 2, and 5 et seq., the present OFF VENT NC (OVNC) nasal apparatus system 10 includes a cannula 12 with oxygen inlet nozzles 14a, 14b on left and right ends. The inlet nozzles 14a, 14b connect to oxygen tubing which delivers inlet oxygen towards the nostrils in a manner corresponding to a standard NC. In the present embodiments the nasal apparatus 10 includes air entrainment windows 20a, 20b at respective locations where the oxygen tubing nozzles 14a, 14b meet the nasal apparatus 10. As evident from FIG. 2, for example, the air entrainment windows 20a, 20b include one or more support structures 22a, 22b that support the nozzles 14a, 14b while open to the ambient environment and thereby define a flow passage for entraining inlet oxygen for mixing and diffusing with room air from the ambient environment.

With ongoing reference to FIGS. 1, 2, and 5 et seq., tapered tubes 24a, 24b are respectively provided downstream of the entrainment windows 20a, 20b. In FIG. 2 the tapered tubes 24a, 24b are discrete components but in FIG. 1 and other embodiments, the tapered tubes 24a, 24b are internal passages within the body of the cannula 12. Each of the tapered tubes 24a, 24b have an air flow passage that is narrowed to utilize the venturi effect to create a high velocity/pressure drop that causes entrainment of air mixed and diffused with oxygen to thereby deliver a high flow of oxygen to the patient's nostrils during inhalation. The air entrainment windows 20a, 20b are composed of soft, silicone-like like material such as a thermoplastic elastomer (TPE) or other soft material to form one-way valves (such as umbrella valves or the like) that allow for the passage of air only during inspiration.

With ongoing reference to FIGS. 1, 2, and 5 et seq., the venturi effect occurs in both nozzles 14a, 14b that connect the oxygen tubing to the nasal apparatus 10 at the side of each of the nostrils. The tapered tubes 24a, 24b define a diffusion chamber enclosed in the nasal apparatus 10. In some embodiments a system of baffles and other geometric designs are used to direct the airflow as needed. The nasal apparatus 10 can contain inhalation passageways 30a, 30b that connect to respective nasal pillows 32a, 32b which contact the nostrils during inhalation. Exhalation passageways 34a, 34b are vents that allow for the passage of air during exhalation. As shown in FIG. 1, these inhalation passageways 30a, 30b and exhalation passageways 34a, 34b can be separate parallel passageways or they can be concentric as shown in FIG. 2. An additional capnography tubing or port 40 can be provided that connects the nasal apparatus 10 to a capnography machine for the monitoring of end tidal CO2 in the exhaled breath of the patient. The capnography tubing or port 40 includes a standard ISO luer connector at the other end for the connection to capnography equipment.

With further reference to FIGS. 1, 2, and 5 et seq., preferred embodiments of the present OVNC system 10 will contain an adjustable PEEP valve 50 that uses a spring-like mechanism 52 (or other similar mechanism) to allow for the passage of less or more air during exhalation, creating the PEEP like effect. This mechanism 52 includes a dial 54 that can be adjusted to control the amount of air flowing through the vents. Typical HFOT systems can deliver flows of up to 60 L/min of fresh gas and claim a PEEP effect of 1 cmH2O for every 10 L/min of fresh gas being delivered. The present OVNC system 10 will typically have high flows of up to 60 L/min or more during inhalation via the venturi effect, and additional fresh gas flow (minus the proximal air entrainment) through the gas delivery tubing during exhalation, so this will create a typical constant PEEP of 1 cmH2O from the fresh gas flow, with the rest of the PEEP created by the amount of air being vented through the vents during exhalation.

As shown, for example, in FIG. 2, in alternate embodiments of the OVNC system 10 the venturi entrainment occurs through one side of the nasal apparatus where the oxygen tubing meets the apparatus, through a one-way valve 60, with the flow then directed towards both nostrils, and the other end of the apparatus containing vents that allow for the expulsion of air during exhalation. This side might also contain a PEEP valve 50 that can be adjusted to control the amount of air flowing through during exhalation. In alternate embodiments of the OVNC, a cushion made of HME filter material encases the nasal apparatus for viral/bacterial filtration and for the conservation of heat and moisture to supplement the humidifier being used in the system.

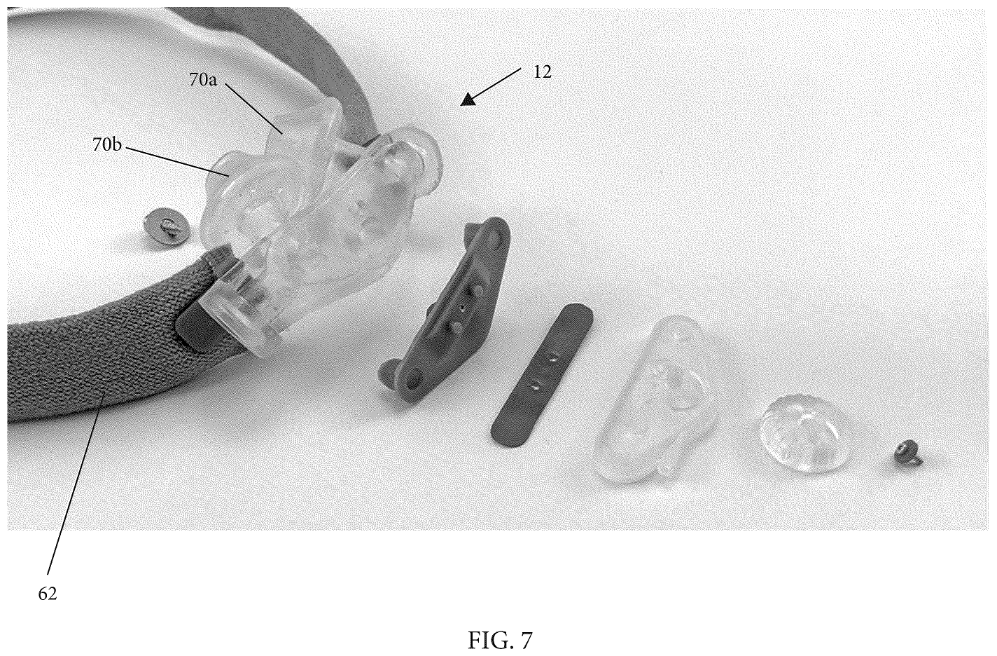

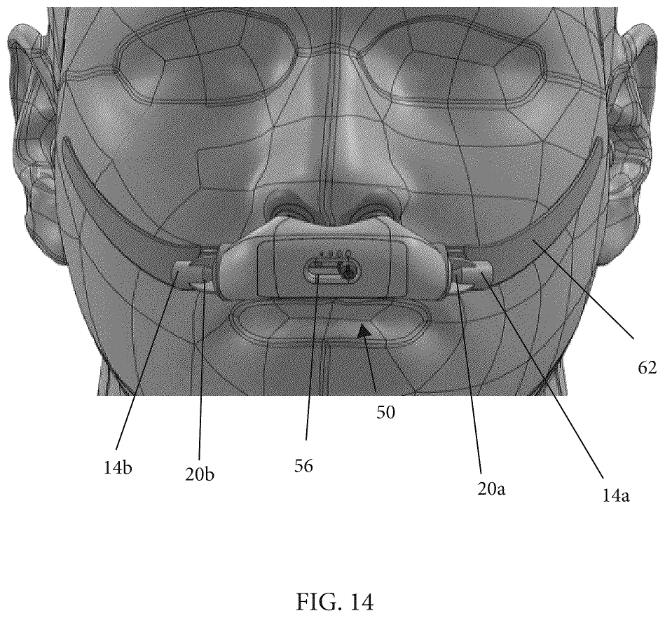

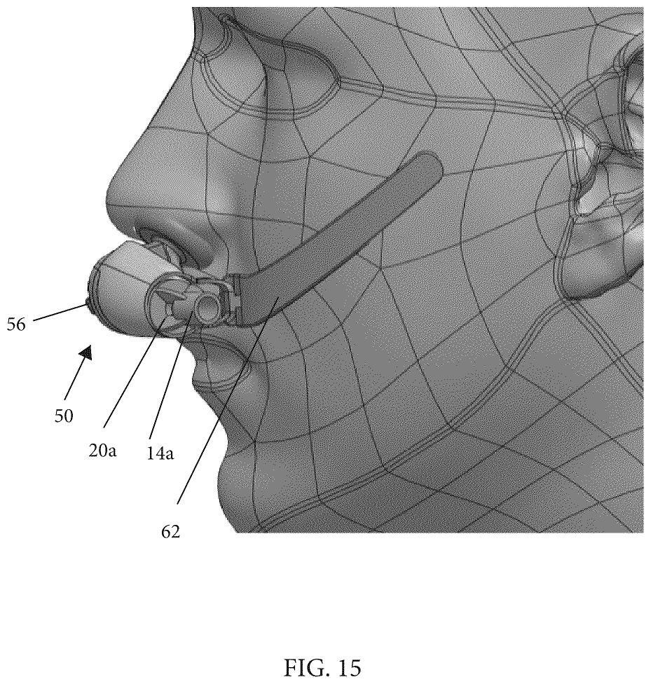

In preferred embodiments of the OVNC system 10, there is an integrated circuit including of a battery, microcontroller etc., to control the diameter of the nozzle of the oxygen supply (where it meets the nasal apparatus), the entrainment window during the venturi effect, and the window for the exhalation of air. In preferred embodiments of the OVNC 10, as shown in FIGS. 7 and 14-16, the nasal apparatus has a head strap 62 for secure attachment to the head of the patient.

The HFNC disclosed herein distinguishes itself from other HFNC systems by using the entrainment of air that occurs at the nasal apparatus, or close to it, to deliver the high flow of fresh gas required to stent airways open. By entraining air through the apparatus, this precludes the need for more robust breathing circuits required to transfer high flowing heated and humified gas to the nostrils. Also, by entraining room air, especially with the augmentation of that air with the WOSHER cap shown in of the present inventor's aforementioned U.S. Pat. No. 11,399,912, the entrained air can be humidified using inexpensive humidification systems, like bubble humidifiers. This system can be used with the oxygen wall supply in hospitals, transport cylinders, oxygen concentrators, ventilators, or any system available for the delivery of fresh gas oxygen or air.

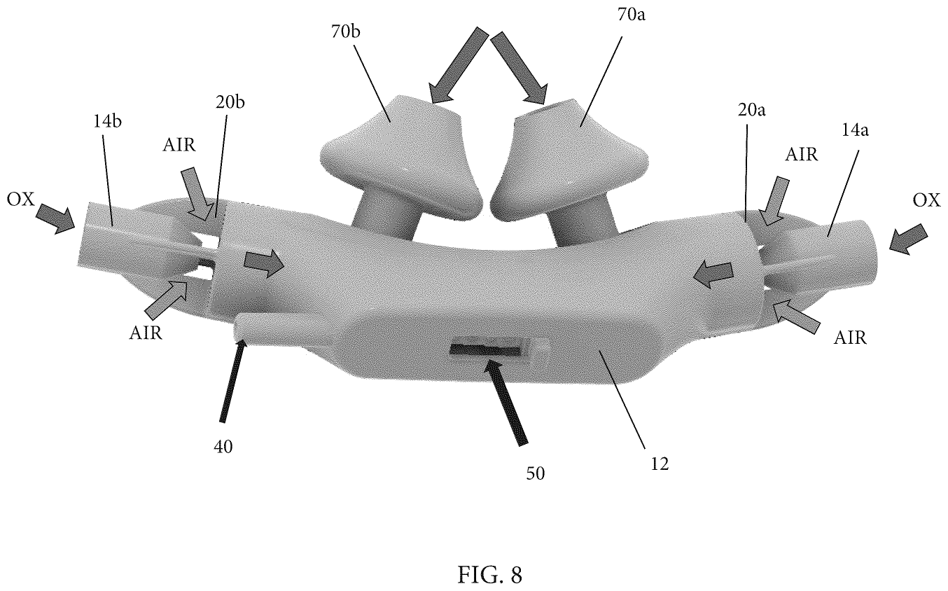

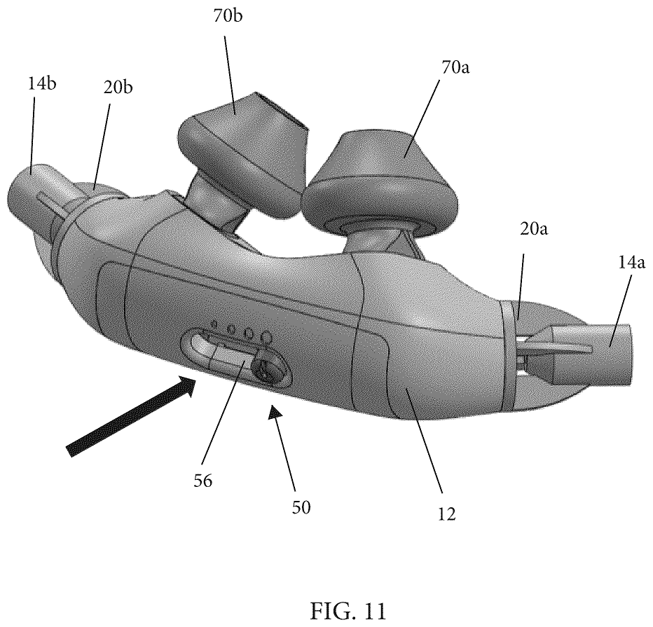

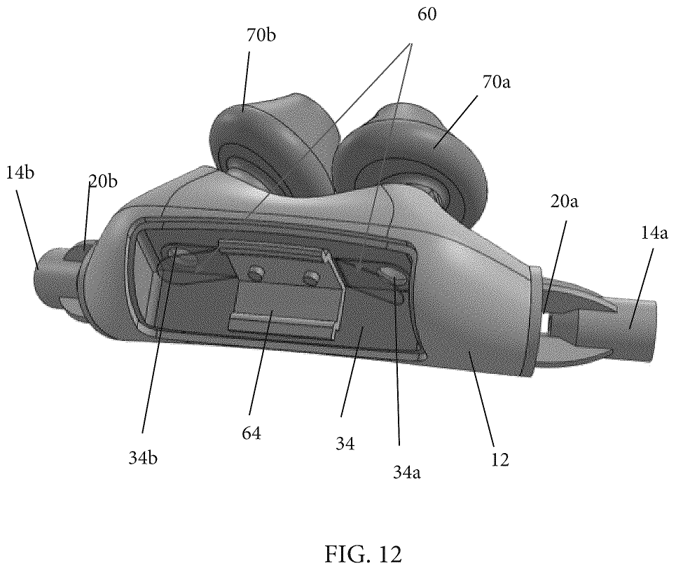

As shown in FIGS. 8, 11, 14, 15 and 16, exemplary embodiments of the present OFF-VENT Airway Adapter include an airway connector lumen, with a perpendicular air entrainment window, either vertical or placed at some angle, with an adjustable entrainment window, and a one-way valve 60 that allows fresh gas to only flow into the system during inspiration. An adapter for a slow, diffusing, possibly heated and humidified, mist of oxygen (the WOSHER) can be attached to this air entrainment window to augment the air that is being drawn through it. The fresh gas coming from the supply flows through the lumen in one direction through a nozzle that forces a pressurized amount of the fresh gas through a constriction that leads to a higher velocity of gas, a pressure drop, and an entrainment of air through the window. In preferred embodiments, this fresh gas is flowing through a tube that is within a larger diameter tube that allows for the circulation of air to and from the patient. The combined gas then flows through a diffusion window through the pressure proof cap into the present respirator mask and into the patient. During exhalation, the air coming from the patient is stopped from flowing back through the air entrainment window by the one-way valve and flows out of the system through an adjustable PEEP valve 50, or into a reservoir bag. The build up of exhaled gas in this reservoir bag causes the opening of an adjustable pressure-limiting (APL) valve, included in the PEEP valve 50, and the exhaustion of exhaled air into the atmosphere.

In preferred embodiments, there is a scavenging leak feature built into the

PEEP/APL valves 50 to prevent the buildup of excessive pressure in the system. In preferred embodiments, there is a manometer attached to the PEEP/APL valve 50 for the measurement of pressure in the entire system.

In alternate embodiments, the oxygen tubing that delivers oxygen to both the main oxygen supply and the WOSHER comes from the same oxygen supply that includes a splitter capable of delivering the same or differential oxygen flow into both the main stem and the WOSHER. In preferred embodiments, there is an extra accessory oxygen supply downstream of the air entrainment window where extra fresh gas can be supplied to the system to enable washout of extra dead space gas and alveolar gas, supplemental oxygen delivery, and the addition of extra PEEP to the system.

In preferred embodiments, a HME filter with a luer connector is placed between the OFF-VENT adapter and the connector on the pressure-proof cap where the system is connected to the patient, for preserving heat and moisture in the system, filtering out respiratory particles both going into the system and coming out of it, and the monitoring of EtCO2 during anesthesia and other use cases. The addition of an HME adapter may be necessary to maintain optimal temperature and humidity in the system during longer duration therapy. In preferred embodiments, there is a seal-scaling access port on the pressure holding, leak-proof cap that allows the introduction of medical instruments or other accessories for patient care. This access port contains a cover, attached via a lanyard that prevents leakage in the system when the port is not being accessed.

In alternate embodiments, the leak-proof cap that is attached to the present respirator contains two or more connectors that can be connected to the OFF-VENT adapter and other airway accessories, either directly or through other airway accessories. In alternate embodiments, the PEEP valve 50 is separated from the rest of the OFF-VENT adapter with the PEEP valve 50 attaching to one of the connectors, and the OFF-VENT adapter attaching to the other(s). Further, to the extent the present application discloses a method, it is contemplated that a system of apparatuses configured to implement the method are also within the scope of the present disclosure.

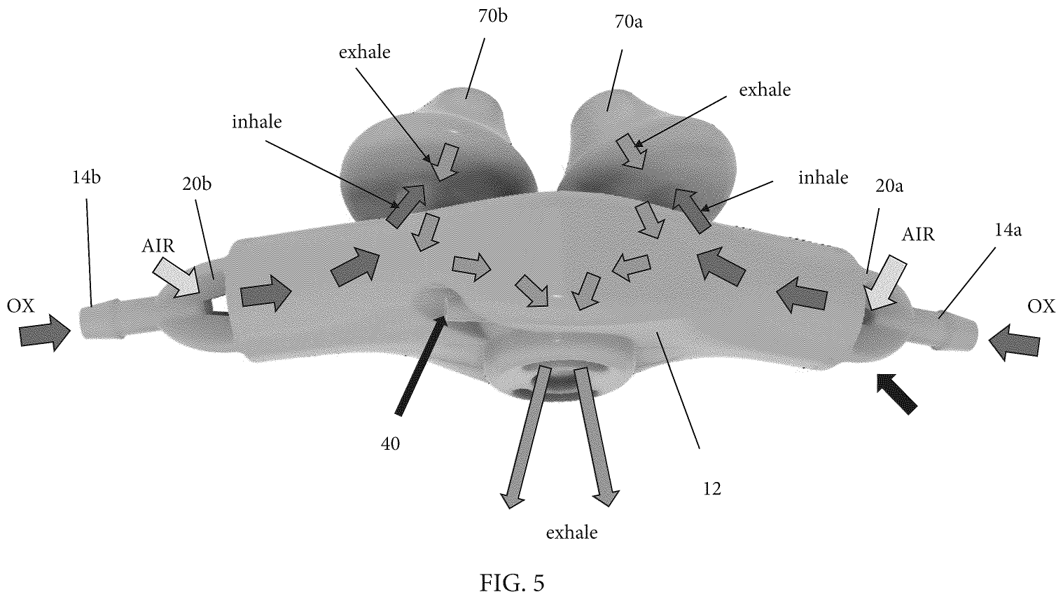

With reference to FIGS. 2 and 5 et seq., the gas from the delivery tubing is guided by a series of baffles in the nasal apparatus into bifurcated nasal cones, prongs, or similar nasal interface, with one chamber (inhalation chamber 30a, 30b) of the bifurcation being contiguous with the airflow from the gas delivery tubing, and the other chamber (exhalation chamber 34a, 34b) contiguous with a different pathway including a one-way valve 60 and an air exit chamber containing a regulatable PEEP valve 50. During inhalation, the user breathes in the gas from the delivery tubing, including the air being entrained from the venturi adapter 24a, 24b through the inhalational chamber 30a, 30b of the bifurcated nasal interface, and the one-way valve 60 in the exhalation chamber 34a, 34b shuts off any air entrainment from the PEEP valve 50 by the negative inspiratory effort of the user, isolating the inhalation chamber 30a, 30b. When the user exhales, they breathe against the gas coming from the inhalation chamber 30a, 30b, which continues to wash out dead space in the upper airway, while the air being breathed out is directed through the exhalation chamber 34a, 34b, through the open one-way valve 60, and through the regulated PEEP valve 50 that can be adjusted to control the rate of air going out, thus the positive end expiratory pressure. In preferred embodiments, the gas delivery inlet into the venturi entrainment windows 20a, 20b on either side of the central nasal apparatus can accommodate different inserts, metal, plastic, or otherwise, that can vary the diameter of the gas delivery inlet to provide various gas velocities, pressure drops, thus various air entrainment ratios or capacity. In alternate embodiments, inserts can also be placed on the other side of the gas inlets 14a, 14b (in the entrainment windows 20a, 20b) to adjust the volume of the air entrainment windows 20a, 20b to vary the amount and/or speed of the air being entrained. In preferred embodiments, the air exit chamber has a tubing or some kind of connector (luer or otherwise) for a capnometry tubing so that CO2 can be measured during breathing.

In alternate embodiments, the nasal apparatus is integrated with electronic

components for power, control, communication and otherwise, including flow sensors and pressure sensors, and an electronic PEEP valve, like a piezo iris diaphragm, or other electronic valve, that can be remotely controlled or automated to self-regulate based on flow and pressure measurements, user and environmental parameters or otherwise. Software, which may be overlayed with artificial intelligence, and which may or may not be tied to an application interface on a computer, can be paired with the electronic system in the nasal apparatus for more functionality.

In the alternate embodiments of FIG. 2, the venturi chambers (i.e., tapered tubes 24a, 24b) on either side of the nasal apparatus are detachable from the center nasal apparatus, allowing connectors of different venturi entrainment windows and gas delivery diameters to be connected to the center apparatus, including differing oxygen: air entrainment ratios (i.e., 15:45, 15:30, 15:15, 40:20 etc.). Connectors could be of differing mechanisms and allow for electronic configuration for detection of the type of connector attached. Connectors could also be of different geometries, allowing for connectors that angle downwards towards the wearer's lips-so as to fit within the perimeters of face masks that can provide more gas, humidified or otherwise, especially oxygen, and nebulized medications etc. that augment the air being entrained—or come straight out towards the cheekbones, or of numerous other designs and angles.

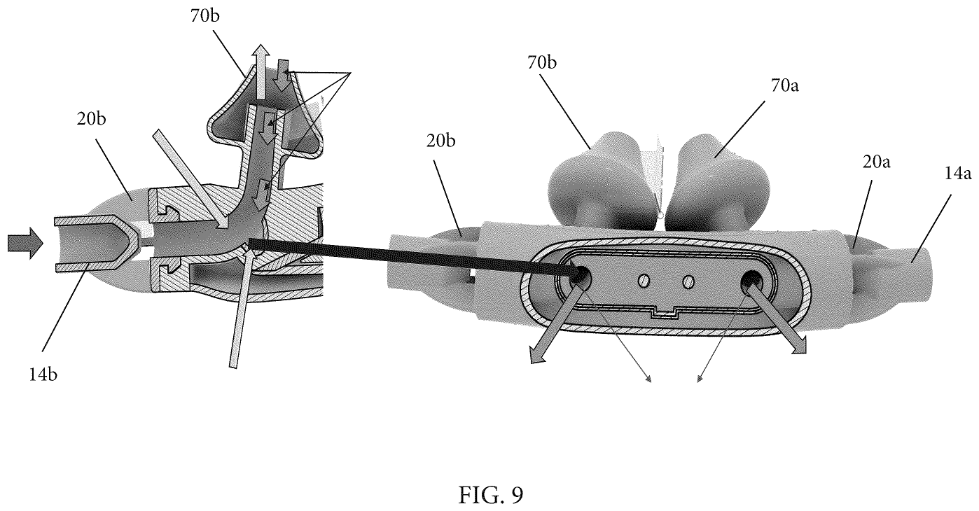

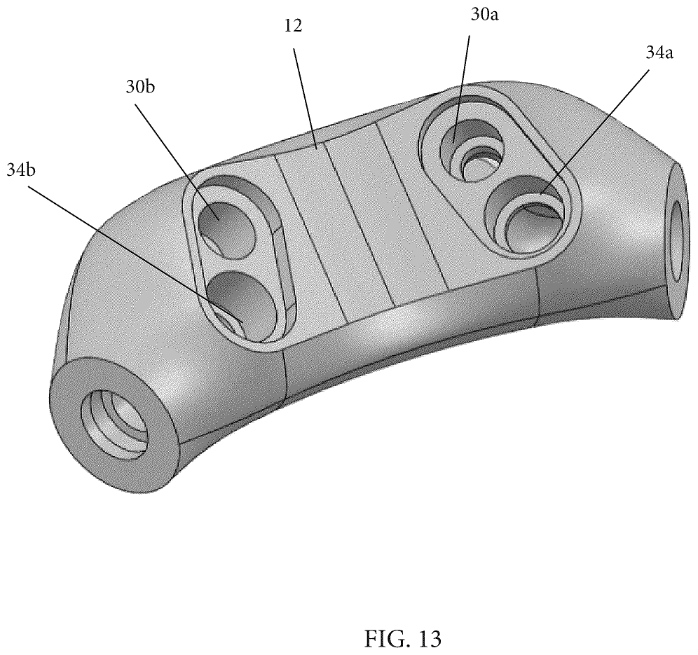

In alternate embodiments such as FIGS. 5-15, the gas delivering tubes, venturi entrainment and diffusion chambers are made of a soft material, like silicone or PVC, that is enclosed by a hard shell, such as polycarbonate, polypropylene etc., that houses the bifurcated nasal prongs, which cover the inhalation and exhalation chambers of the soft tubing. The hard shell of the nasal cannula 12 that houses the one-way valve 60 and the PEEP valve 50 forms contiguous pathway with the exhalation chamber of the soft tubing. While oxygen as described herein, any suitable gas can be delivered to a patient and bifurcation means a division of said chamber or apparatus.

In an alternate embodiment, a tube can be provided inside a bigger tube that delivers fresh gas to the system and built to accommodate insertions of various diameters and flow constriction capabilities, so as to vary the velocity of the gas being delivered, and the pressure drop, to provide various forms of air entrainment through the windows of the OFF VENT adapter. In preferred embodiments, the dial 54 is placed parallel to the windows of the OFF VENT adapter to regulate the air entrainment windows, corresponding to different gas delivery inserts.

In preferred embodiments, the circuit described hereinabove can be used with the OFF VENT NC described previously or with a nasal mask or any other patient interface. When used with the OFF VENT NC, the gas flow and air entrainment work as described, but the exhalation and PEEP functionality take place through the nasal apparatus, excluding the breathing bag and the APL/PEEP valve 50 from the equation. When used with the nasal mask, or other positive pressure patient interface, the breathing bag and the APL/PEEP valve 50 play a role as previously described.

Further to the description of FIG. 2 hereinabove, an embodiment of the OFF-VENT NC system as described hereinabove includes inhalation chambers (i.e., inhalation passageways 30a, 30b that are coaxially surrounded by a plurality of exhalation passageways 34a, 34b in the form of multiple axial orifices on the perimeter (preferably 6 surrounding each passageway) that divert the exhaled air into two respective exhalation chambers that create a contiguous pathway with the one-way valve 60 and the PEEP/pressure release valve 50. The nasal apparatus is preferably made of silicone or a similar soft, rubber type material. The inhalation chamber, and the surrounding axial orifices, can accommodate a nose cone 70 (as shown in other figures) that connects to the wearer's nostrils and fully or partially seals on the nostrils, forcing the airflow to move through the apparatus. The chosen material has enough compliance to accommodate various patient anatomies. In an alternate embodiment, the apparatus can be made of a harder plastic material.

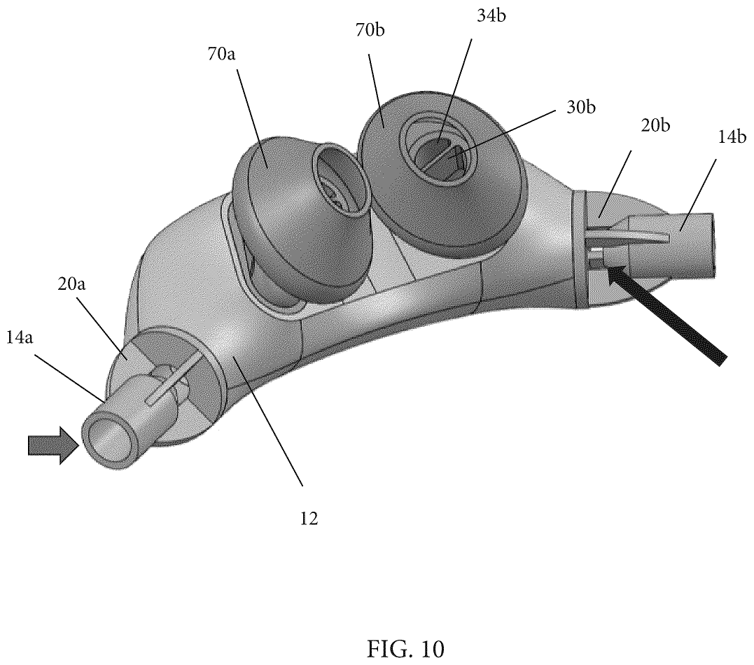

As shown in FIGS. 10, 11, and 12, the present OFF-VENT NC system 10 includes a main body of the nasal apparatus made of a soft material like silicone, and includes two lateral slots that accommodate the insertion of the gas inlet adapters (both air entraining and non-air entraining), and a platform on the superior side of the nasal apparatus including 4 slots-two posterior slots respectively accommodating the inhalation passageways 30a, 30b of the orbital nasal pillows 70a, 70b, and two anterior slots accommodating the exhalation passageways 34a, 34b of the orbital nasal pillows 70a, 70b. The orbital nasal pillows 70a, 70b are interchangeable, with the main body of the nasal apparatus accommodating different sizes. In an alternate embodiment, the inhalation and exhalation passageways 30a, 30b, 34a, 34b to the main body of the apparatus are reversed, with the inhalation passageways being anterior and the exhalation passageways being posterior. Other configurations are possible as well. Orientation as referred to herein refers to the nasal apparatus as worn by the user, with the posterior portion touching the face, the anterior portion (containing the PEEP valve 50) pointing away from the user to the front, and the superior portion being directed towards the nostrils.

With further reference to FIGS. 10, 11, and 12, the orbital nasal pillows 70a, 70b are overmolded (or some other form of attachment) to rigid connectors that separate the inhalation passageways 30a, 30b from the exhalation passageways 34a, 34b. These rigid connectors press fit (or some other form of attachment) into the body of the main nasal apparatus. The orbital nasal pillows are compressible to allow for better fit into the nostrils of the user. The orbital nasal pillows also allow for rotation around the axis of the rigid connectors for flexibility and easier manipulation for fit and convenience. In preferred embodiments, the orbital nasal pillows are bifurcated on the insides to separate the inhalation and exhalation passageways. In alternate embodiments, the nasal pillows are not bifurcated on the inside and can have other configurations of separation or be one passageway.

In the embodiments of FIGS. 10, 11, and 12, the platform of inhalation and exhalation passageways 30a, 30b, 34a, 34b on the nasal cannula body 12 are made of rigid ports that accommodate the orbital nasal pillows 70a, 70b via a press fit, or other type of coupling mechanism. These rigid port fixtures are made of polycarbonate (or other type of rigid material) and are overmolded with silicone or another type of soft, medical grade material, like TPEs. This overmolded soft piece creates the body of the nasal apparatus, the rest of the inhalation and exhalation passageways, and the housing for the one-way valve 60 and the PEEP valve 50.

As particularly shown in FIG. 12 the one way valve 60 can be formed of a thin layer membrane of soft material like silicone, polyurethane, TPEs, or other soft, medical grade material, with a fixture 64 in place holding the one-way valve 60 against the exhalation passageways 34a, 34b. Both ends of the soft membrane close a unitary internal exhalation passageway 34 during inhalation, to prevent unnecessary air entrainment, and open during exhalation to allow air to flow through into the exhalation chamber 34 and the PEEP valve 50. In preferred embodiments, a sliding mechanism 56, with a pointer dial, and markings to connotate the amount of air opening, serves as the PEEP valve 50, regulating the exhalation airflow, and building up positive pressure in the pulmonary system.

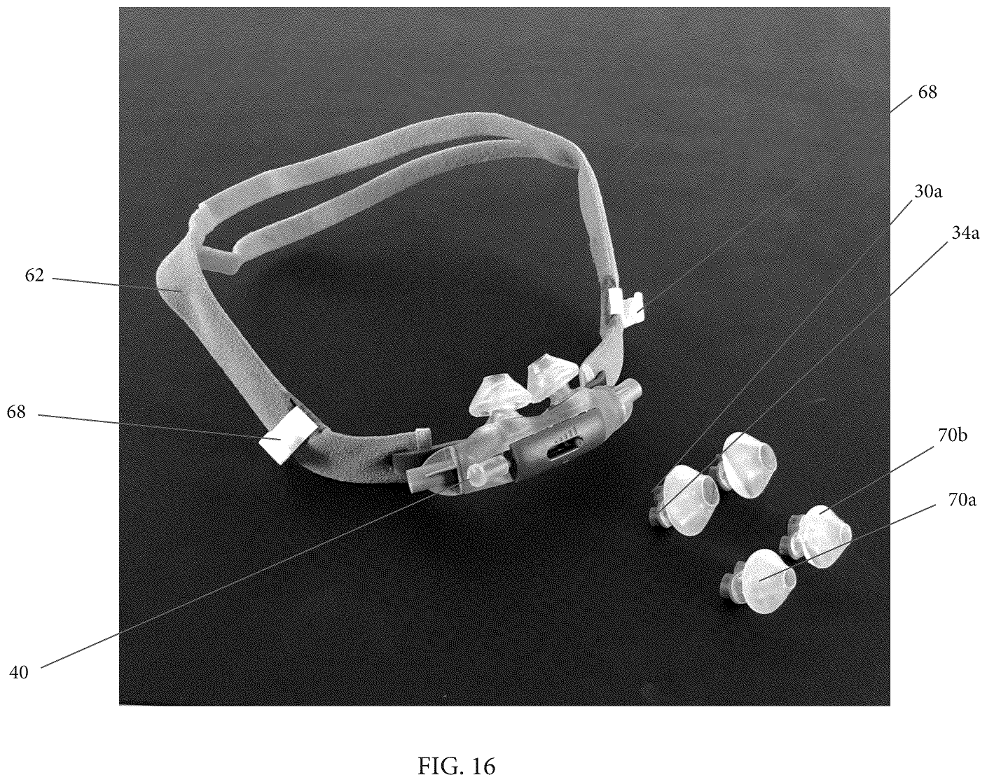

As shown in FIG. 16, connectors that link the main body 12 of the nasal apparatus to the head straps 62, providing firmness, are made of polycarbonate, or similar plastic, and overmolded with soft silicone to make up the body 12 of the nasal apparatus. These connectors then slip into slots on the head gear, providing some sturdiness and easier manipulation for the users. In preferred embodiments, these connectors contain fixtures for the addition of hook 68 that serve as attachment spots for the gas tubing, preventing contact with the skin of the users to prevent pressure injuries and other forms of discomfort.

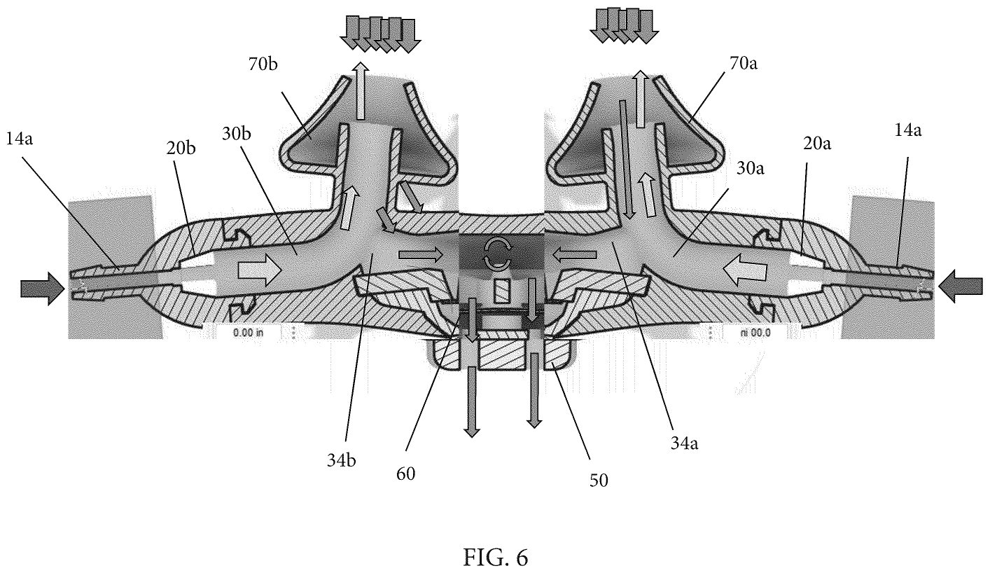

As shown in FIGS. 5 and 6, both inhalation and exhalation occur through a common passageway in the nasal apparatus that connects the slots that accommodate the gas inlet adapters to the orbital nasal pillows 70a, 70b. The orbital nasal pillows 70a, 70b make a uniform piece with the soft body 12 of the nasal apparatus made of silicone or a similar, soft, medical grade material. The soft body 12 of the main apparatus provides housing for rigid fixtures that make the exhalation chamber and the PEEP valve 50. In certain embodiments, the exhalation windows 20a, 20b come off the common passageway in an anterior (facing away from the wearer) orientation on either side, forming bilateral passageways that lead to the one-way valve 60 formed of a soft, thin membrane, the exhalation chamber, and the PEEP valve 50 at the anterior portion of the nasal apparatus. In alternate embodiments, the exhalation chamber 34 and PEEP valves 50 are oriented on the inferior portion of the apparatus, towards the mouth.

In alternate embodiments, an initial rigid component connects to the main body of the apparatus via plug in fixtures (or other forms of coupling) to create the exhalation passageways. This rigid piece contains nipples for the attachment of the soft, thin membrane that is the one-way valve. A second rigid piece sandwiches the one-way valve to create the exhalation pathway. This rigid piece contains both the capnometry port 40 and the opening for the dial 56 that controls the PEEP valve 50. This dial 56 is attached to a second adjoining rigid piece via a screw, or some other coupling mechanism. In certain embodiments, the dial 56 and PEEP valve assembly 50 are configured at the anterior portion of the nasal apparatus, directing air away from the patient and towards the atmosphere. In alternate embodiments, they are configured at the inferior portion of the nasal apparatus, directing air towards the mouth.

With reference to an exemplary nasal cannula embodiment depicted in FIGS. 10-16, the sliding mechanism 56 can alternatively include a divider that separates the right and left nostrils. This embodiment does not include a one-way valve 60. The inlet nozzles 14a, 14b are not detachable but instead incorporate flow inlet tubing integral with the main body of the device.

With ongoing reference to FIGS. 10-16, the aforementioned structure of the exemplary embodiment employs unidirectional flow in which gas flows out of only one of the nostrils when the patient is breathing out, rather than out of both of them. This unidirectional flow has been shown to have some benefits in increasing airway pressure and clearing out CO2 faster from the airways. This is accomplished by restricting the flow out of one nostril more than the other; and increasing the flow into one nostril more than the other. Preferentially increasing the flow into one nostril while concurrently restricting the flow out of it will lead to flow mainly going out of the other nostril. The first part of this is accomplished in the present embodiment by having the PEEP valve 50 in a restrictive setting on one nostril and more open on the other. The second part of this concerns how flow is directed into either side of the nasal cannula.

With further reference to FIGS. 10-16, the aforementioned first part works like

the chamber of a revolver in which there are 4 pathways (two inhalation pathways at the top, and two exhalation pathways at the bottom). These are connected to a circuit with an inlet of gas, and a possible outlet, with a flow controller in the middle containing multiple apertures. The apertures are aligned with the pathways. The inlet of gas has a Y connector that directs the common inlet of gas into two branches on the Y that lead to the flow controller and the inhalation pathways. In the default position, the apertures are fully open when aligned with the 4 pathways. With one twist to the right, a constricting aperture is aligned with one of the inhalation pathways, restricting gas into it while keeping the other inhalation pathway open. Concurrently, the opposite exhalation pathway on the bottom is now aligned with an aperture that partially or fully occludes it. The reverse happens with a twist to the left. Safety mechanisms are in place to prevent overturning in either direction. The aforementioned second part employs the same concept explained above but with a horizontally sliding flow direction rather than a revolving one. A clamp (roller, slider etc.) can be used to restrict the flow into one pathway while maintaining/increasing the flow in the other pathway.

Numerous embodiments have been described herein. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

What is claimed:

1. A nasal apparatus system, comprising:
a cannula having at least one oxygen inlet nozzle connected to oxygen tubing which delivers inlet oxygen towards nostrils of a patient;
at least one air entrainment window positioned on the cannula to support the oxygen inlet nozzle while open to the ambient environment and thereby define a flow passage for entrainment of inlet oxygen from the nozzle for mixing and diffusing with room air from the ambient environment to produce a mixed flow; and
at least one tapered tube provided downstream of the entrainment window for receiving the mixed flow from the entrainment window and having an air flow passage that is narrowed from a first diameter to a second diameter to utilize a venturi effect to create a high velocity/pressure drop that causes the entrainment of the mixed flow to thereby deliver a high flow of oxygen to the patient's nostrils during inhalation.

2. The nasal apparatus system of claim 1, wherein the cannula comprises first and second oxygen inlet nozzles formed with respective first and second air entrainment windows that cooperate with respective first and second tapered tubes to deliver first and second mixed flows to the nostrils of the patient.

3. The nasal apparatus system of claim 2, wherein the respective first and second nozzles, windows and tubes are configured to deliver the first and second mixed flows to respective left and right nostrils of the patient.

4. The nasal apparatus system of claim 1, wherein the air entrainment window includes at least one support structure to support the oxygen inlet nozzle while opening to the ambient environment.

5. The nasal apparatus system of claim 1, wherein the tapered tube is a discrete component with the air passage formed internally.

6. The nasal apparatus system of claim 1, the tapered tube is an internal component formed within a body of the cannula to define the air passage.

7. The nasal apparatus system of claim 6, wherein the body of the cannula further comprises at least one inhalation passageway connected to at least one respective nasal pillow for contacting the nostrils during inhalation, and further comprising at least one exhalation passageway to allow for passage of air during exhalation.

8. The nasal apparatus system of claim 7, further comprising a one-way valve over the exhalation passageway to allow for the expulsion of air during exhalation.

9. The nasal apparatus system of claim 8, wherein the one way valve is formed of a thin layer membrane of soft material and having a fixture for holding the one-way valve against the exhalation passageway.

10. The nasal apparatus system of claim 7, further comprising an adjustable positive end-expiratory pressure (PEEP) valve configured over the exhalation passageway to allow for variable passage of less or more air during exhalation.

11. The nasal apparatus system of claim 10, wherein the adjustable PEEP valve includes a spring-like mechanism with an adjustable dial to control an amount of air flowing out through the exhalation passageway.

12. The nasal apparatus system of claim 11, wherein the dial is a pointer dial that cooperates with a sliding mechanism having markings to connotate the amount of air opening, for regulating exhalation airflow and positive pulmonary system pressure.

13. The nasal apparatus system of claim 7, wherein the inhalation and exhalation passageways are configured to be separate parallel passageways.

14. The nasal apparatus system of claim 13, wherein the inhalation and exhalation passageways are formed within a generally cylindrical structure divided by a bifurcation so that airflow between the inhalation and exhalation passageways are contiguous.

15. The nasal apparatus system of claim 7, wherein the inhalation and exhalation passageways are configured to be concentric passageways.

16. The nasal apparatus system of claim 15, wherein the inhalation passageway is formed within a central cylindrical structure surrounded by a plurality of coaxial orifices to divert exhalation air.

17. The nasal apparatus system of claim 1, further comprising a capnography port for connecting the nasal apparatus to a capnography machine for the monitoring of end tidal CO2 in the exhaled breath of the patient.
